(19) United States
(12) Patent Application Publication
(10) Pub. No.: US 2012/0270226 A1
(43) Pub. Date: Oct. 25, 2012

(54) MATCHING OF FORENSIC RESULTS

(75) Inventors: Matthew John Barron, Solihull (GB); Martin Bill, Solihull (GB)

(73) Assignee: FORENSIC SCIENCE SERVICE LIMITED, Solihull (GB)

(21) Appl. No.: 13/509,033

(22) PCT Filed: Nov. 10, 2010
(86) PCT No.: PCT/GB10/02070

§ 371 Date: Jul. 9, 2012

(30) Foreign Application Priority Data
Nov. 10, 2009 (GB) 0919686.6
Nov. 12, 2009 (GB) 0919766.6
Jan. 22, 2010 (GB) 1000990.0

(51) Int. Cl. G06F 17/18; C12Q 1/68

(52) U.S. Cl. 435/6.12; 702/19

(57) ABSTRACT

A method of analysing and comparing a test sample with another sample is provided, wherein: the test sample is analysed, the analysis producing test sample analysis data set; the test sample analysis data set is processed to give a test sample results data set; defining a search term relating to the test sample results data set; obtaining the another sample to compare with the test sample; comparing the another sample result with the search term to inform on the another sample being a potential match with the test sample.

This invention is concerned with improvements in and relating to matching of forensic results, and in particular, but not exclusively, to matching a result from the analysis of a DNA sample to one or more stored results.

In many situations, particularly in forensic science, there is a need to consider one piece of evidence against one or more other pieces of evidence.

For instance, it may be desirable to compare a sample collected from a crime scene with a sample collected from a person, with a view to linking the two by comparing the characteristics of their DNA. This is an evidential consideration. The result may be used directly in criminal or civil legal proceedings. Such situations include instances where the sample from the crime scene is contributed to by more than one person.

In other instances, it may be desirable to establish the most likely matches between examples of characteristics of DNA samples stored on a database with a further sample. The most likely matches or links suggested may guide further investigations. This is an intelligence consideration.

In both of these instances, it is desirable to be able to express the strength or likelihood of the comparison made, a so called likelihood ratio, particularly for an evidential consideration or a probability for an intelligence consideration.

When making the comparison, particularly in the intelligence consideration scenario, recent developments have significantly increased the number of results within a data set for a sample which must be compared with pre-existing results. The large number of results in the data set and the enormous number of results on a database, for instance, can give rise to computational problems in terms of resources and/or time. In making the transition to the data sets having the increased number of results and being able to compare those, there is also a problem with still being able to make use of the enormous number of results of the older format.

The present invention has amongst its possible aims to provide improved matching of results. The present invention has amongst its possible aims to provide a matching of results using existing matching logic, whilst ideally considering both old and new style data sets. The present invention has amongst its possible aims to reduce the computational problems with applying matching logic to the new style data sets. The present invention has amongst its possible aims to provide for a composite database incorporating both old and new style data sets.

According to a first aspect of the invention there is provided a method of comparing a test sample with another test sample, wherein:

the test sample is analysed, the analysis producing test sample analysis data set;

the test sample analysis data set is processed by a computer implemented step to give a test sample results data set, the test sample results data set including one or more test sample results, a test sample result including an identity value for one or more of the identity variables considered in the analysis, the possible identity values including a constrained identity value, the constrained identity value being two or more of the possible identity values and less than all of the possible identity values;

defining, in a computer implemented step, a search term from one or more of the test sample results, the defining of the search term involving the replacement of one or more of the constrained identity values with an unconstrained identity value, the unconstrained identity value being all the possible identity values;

obtaining, in a computer implemented step, the another sample to compare with the test sample, the another sample being in the form of an another sample result data set, the another sample result data set including one or more another sample results, an another sample result including an identity value for one or more of the identity variable considered in an analysis of that another sample;

comparing, in a computer implemented step, the another sample result with the search term, the another sample result complying with the requirements of the search term and thereby providing a potential match between the another sample and the test sample or the another sample result not complying with the requirements of the search term and thereby not providing a potential match between the another sample and the test sample;

allocating, in a computer implemented step, a probability and/or a probabilistic weighting to one or more of the potential matches;

ranking, in a computer implemented step, the one or more potential matches provided with a probability and/or a probabilistic weighting according to the value of the allocated probability and/or probabilistic weighing.

According to a second aspect of the invention there is provided a method of comparing a test sample with another test sample, wherein:

the test sample is analysed, the analysis producing test sample analysis data set;

the test sample analysis data set is processed to give a test sample results data set;

defining a search term relating to the test sample results data set;

obtaining the another sample to compare with the test sample;

comparing the another sample result with the search term to inform on the another sample being a potential match with the test sample.

The method may provided that the test sample analysis data set is processed by a computer implemented step.

The method may provide that the test sample results data set includes one or more test sample results. The method may provide that a test sample result includes an identity value for one or more of the identity variables considered in the analysis. The method may provide that the possible identity values include a constrained identity value. The method may provide that the constrained identity value as being two or more of the possible identity values and less than all of the possible identity values.

The method may include the defining a search term being a computer implemented step. The method may include defining the search term from one or more of the test sample results. The method may include the defining of the search term involving the replacement of one or more of the constrained identity values with an unconstrained identity value. The unconstrained identity value may have all the possible identity values.

The method may include the obtaining the another sample being a computer implemented step. The another sample may be in the form of an another sample result data set. The another sample result data set may including one or more another sample results. An another sample result may include an identity value for one or more of the identity variable considered in an analysis of that another sample.

The method may include the comparing being a computer implemented step. The method may include that where the another sample result complies with the requirements of the search term, that thereby indicates a potential match between the another sample and the test sample. The method may include that where the another sample result does not comply with the requirements of the search term, that thereby indicates there is not a potential match between the another sample and the test sample.

The method may include the allocating being a computer implemented step. The method may include allocating a probability and/or a probabilistic weighting to one or more of the potential matches.

The method may include the ranking being a computer implemented step. The method may include ranking the one or more potential matches provided with a probability and/or a probabilistic weighting according to a value, particularly the value of the allocated probability and/or probabilistic weighing.

The first and/or second aspects of the invention may include the following features, options or possibilities or those set out elsewhere in this document.

The test sample may be a sample from an unknown source. The test sample may be a sample from a known source, particularly a known person.

The test sample may be contributed to by a single source. The test sample may be contributed to by an unknown number of sources. The test sample may be contributed to by two or more sources. One or more of the two or more sources may be known, for instance the victim of the crime.

The test sample may be considered as evidence, for instance in civil or criminal legal proceedings. The evidence may be as to the relative likelihoods, a likelihood ratio, of one hypothesis to another hypothesis. In particular, this may be a hypothesis advanced by the prosecution in the legal proceedings and another hypothesis advanced by the defense in the legal proceedings.

The test sample may be considered in an intelligence gathering method, for instance to provide information to further investigative processes, such as evidence gathering. The test sample may be compared with one or more previous samples or the stored analysis results therefore. The test sample may be compared to establish a list of stored analysis results which are the most likely matches therewith.

The another sample may be a sample from an unknown source. The another sample may be a sample from a known source, particularly a known person.

The another sample may be contributed to by a single source. The another sample may be contributed to by an unknown number of sources. The another sample may be contributed to by two or more sources. One or more of the two or more sources may be known, for instance the victim of the crime.

The another sample may be one another sample from amongst at least 100, preferably at least 1000, more preferably at least 10000 and ideally at least 1000000 such another samples stored in a data base. The another samples may be stored as another sample result data sets and/or another sample results.

The another sample may have previously been analysed and/or been added to the database.

The comparing may be used to considered evidence, for instance in civil or criminal legal proceedings. The comparing may make a comparison as to the relative likelihoods, for instance a likelihood ratio, of one hypothesis to another hypothesis. The comparing may make a comparison as to the relative likelihoods of the evidence relating to one hypothesis to another hypothesis. In particular, this may be a hypothesis advanced by the prosecution in the legal proceedings and another hypothesis advanced by the defense in the legal proceedings. The likelihood ratio may be of the form:

$$LR = \frac{p ( c , gs \vert {V}_{p} )}{p ( c , gs \vert {V}_{d} )} = \frac{f ( c \vert gs , {V}_{p} )}{f c \vert gs , {V}_{d} )}$$

where
1. c is the test result data set from a test sample, more particularly, the test result data set taken from a sample recovered from a person or location linked with a crime, potentially expressed in terms of peak positions and/or heights and/or areas;
2. gs is the another sample result data set, more particularly, the another result data set taken from a sample collected from a person, particularly expressed as a suspect's genotype;
3. Vₚ is one hypothesis, more particularly the prosecution hypothesis in legal proceedings stating “The suspect left the sample at the scene of crime”;
4. V_{d} is an alternative hypothesis, more particularly the defense hypothesis in legal proceedings stating “Someone else left the sample at the crime scene”.

The comparing may provide for a step including or providing a Likelihood Ratio, LR. The LR may summarise the value of the evidence in providing support to a pair of competing propositions: one of them representing the view of the prosecution (Vₚ) and the other the view of the defense (V_{d}). The propositions may be:
1) Vₚ: The suspect is the donor of the DNA in the test sample;
2) V_{d}: Someone else is the donor of the DNA in the test sample.

The method of comparing may be used to gather information to assist further investigations or legal proceedings. The method of comparing may provide intelligence on a situation. The method of comparison may be of the likelihood of the information of the test sample result given the information of the another sample result. The method of comparing may provide a listing of possible another sample results, ideally ranked according to the likelihood. The method of comparison may seek to establish a link between a DNA profile from a crime scene sample, a test sample, and one or more DNA profiles stored in a database, an another sample.

The method of comparing may provide a link between a DNA profile, for instance from a crime scene sample, and one or more profiles, for instance one or more profiles stored in a database.

The method of comparing may consider a crime profile with the crime profile consisting of a set of crime profiles, where each member of the set is the crime profile of a particular locus. The method may propose, for instance as its output, a list of profiles from the database. The method may propose a posterior probability for one or more or each of the profiles. The method may propose, for instance as its output, a list of profiles, for instance ranked such that the first profile in the list is the genotype of the most likely donor.

The method may further provide that where one or more potential matches are identified, a decision may be taken. The decision may be to take one or more actions. The method may include performing one or more actions. The method may include a step for deciding to perform one or more actions and/or for instructing the performance of one or more action and/or for performing one or more actions.

The method may further provide that where no potential matches are identified, a decision may be taken and/or an instruction may be given. The decision and/or instruction may be different to the decision taken where one or more potential matches are identified. The decision and/or instruction may be to take one or more actions. The one or more actions may be different to the one or more actions taken where one or more potential matches are identified. The decision and/or instruction may be not to take one or more actions.

The method may include a step for performing one or more actions The method may include a step for performing one or more actions where one or more potential matches are identified. The method may include a step for performing one or more actions where no potential matches are identified.

The method may include a step for collecting a subsequent sample.

The method may include a step for performing an analysis of a subsequent sample.

The subsequent sample may be a subsequent sample taken from the test sample and/or a subsequent sample taken from the same source as the test sample. The subsequent sample may be another part or aliquot of the test sample. The subsequent sample may be a new subsequent sample taken from the same source as the test sample. The source may be an item and/or a location and/or a person.

The subsequent sample may be a subsequent sample taken from the another sample and/or a subsequent sample taken from the same source as the another sample. The subsequent sample may be another part or aliquot of the another sample. The subsequent sample may be a new subsequent sample taken from the same source as the another sample. The source may be an item and/or a location and/or a person.

The subsequent sample may be collected from an item and/or a location and/or a person.

The subsequent sample may be analysed by a physical analysis method. The physical analysis method may include one or more of test sample preparation, purification, amplification, size based separation, electrophoresis, fluorescence or light detection. The analysis method may seek to establish the identity values for present in respect of one or more identity variables st locations in the DNA of the subsequent sample. The one or more identity variables may be the allele or alleles present at a locus. The analysis may establish the one or more identity values present at one or more loci. The analysis may establish the one or more identity values present at one or more loci in terms of allele designation, for instance according to a recognised system of designation according to size. The analysis may establish the one or more identity values present at one or more loci in terms of a constrained range of possibilities, a constrained identity value.

The subsequent sample may be processed using one or more steps of the aspects of the invention and/or according to the aspects of the invention and/or according to any of the options or possibilities set out elsewhere within this application. The subsequent sample may be compared with the another sample and/or with a different another sample and/or with a different subsequent sample.

The method may include a step for moving an item from a first state to a second state. The first state may be a position. The second state may be a position. Preferably the first state is a first position and the second state is a second different position.

The item may be the source of a subsequent sample. The item may be a sample collection container, sample collection device or sample. The item may be a vehicle. The item may be a person. The person may be the source of a sample and/or test sample and/or another sample and/or subsequent sample.

The analysis may include one or more of test sample preparation, purification, amplification, size based separation or electrophoresis. The analysis may seek to establish the identity values present in respect of one or more identity variables at locations in the DNA of the test sample. The one or more identity variables may be the allele or alleles present at a locus. The analysis may establish the one or more identity values present at one or more loci. The analysis may establish the one or more identity values present at one or more loci in terms of an allele designation, for instance according to a recognised system of designation or according to size. The analysis may establish the one or more identity values present at one or more loci in terms of a constrained range of possibilities, a constrained identity value.

The test sample analysis data set may be the output from the instrument performing the analysis. The analysis may be performed by an instrument which is separate from the computer implementing one or more of the subsequent steps.

The processing of the test sample analysis data set to give the test sample results data set may be provided according to the approach detailed in International Patent Publication number WO2009/066067 and/or US Patent Application Publication number US2009/0132173 and/or International Patent Publication number WO2010/116158.

The processing of the test sample analysis data set may include determining one or more or all of the possible combinations of identity values which could have given rise to the test sample analysis data set. The processing of the test sample analysis data set may include determining one or more or all of the possible combinations of allele values which could have given rise to the test sample analysis data set.

The test sample results data set may include one or more or all of the test sample results. The test sample results data set may include one or more or all of the possible combinations of identity values which could have given rise to the test sample analysis data set and/or one or more or all of the possible combinations of allele values which could have given rise to the test sample analysis data set.

A test sample result may be one of the possible combinations of identity values which could have given rise to the test sample analysis data set. A test sample result may be one of the possible combinations of allele values which could have given rise to the test sample analysis data set.

The identity value may be expressed in terms of an allele designation, for instance according to a recognised system of designation or according to size. The identity value may be expressed in terms of a constrained range of possibilities, a constrained identity value, for instance in terms of two or more allele designations.

The identity variables considered in the analysis may be one or more and preferably at least 8 loci considered in the analysis. The identity variables may be capable of heterozygous and/or homozygous variation. The variation may be one or more values selected from a limited number of values, for instance the known range of variation in the alleles for that locus.

The possible identity values may cover the variation possible, for instance one or more values selected from a limited number of values, for instance the known range of variation in the alleles for that locus. The possible identity values may include one or more numerical values. A numerical value may represent a single identity value for an identity variable, for instance an allele value for a locus. The possible identity values may include one or more non-numerical values, for instance a letter designation. A non-numerical value may represent two or more identity values for an identity variable, for instance two or more allele values for a locus. The constrained identity value may be a non-numerical value.

The constrained identity value may have a value equal to two or more of the possible identity values, for instance two or more allele values. The possible identity values may be those which exist or which are known for that identity variable, for instance that locus. The possible identity values may include the value “unknown” for the identity variable. The possible identity values may not be deemed to include the value “unknown” for the identity variable.

One or more restrictions may be applied to the test results before the search term is defined and/or during the defining of the search term.

One of the one or more restrictions may be to exclude one or more test sample results. One of the one or more restrictions may be to exclude one or more test sample results according to a further criteria.

The exclusion or further exclusion may be of test sample results which have an identity value or identity values which are selected to excluded, for instance by the computer and/or the user. The exclusion or further exclusion may be of those test results which do not match the conditioning terms applied. The exclusion or further exclusion may be of those test results which do not have at least one of a selected identity value or values, for instance as set by the computer and/or user.

The test sample results data set may be obtained from the test sample analysis data set by excluding one or more parts of the test sample analysis data set. The one or more parts excluded may be those parts of the test sample analysis data set determined to be caused by sources which are not identity variables, such as non-allelic sources and/or may be caused by sources which are not identity variables, such as non-allelic sources. Not identity values and/or non-allelic sources may arise from outside the test sample and/or from outside of the source of the test sample and/or where the test sample is from a mixture of sources, a source outside of that mixture of sources. One or more parts may be excluded where they are only suspect of being caused by sources which are not identity variables, such as non-allelic sources. Not identity values and/or non-allelic sources may be one or more or all of stutter of alleles, drop in of alleles, contamination of the sample, stochastic events, or other non-allelic sources.

The test sample results data set may include one or more parts of the test sample analysis data set which are not identity variables, such as non-allelic sources, and/or which are suspected of being caused by sources which are not identity variables, such as non-allelic sources.

The processing of the test sample analysis data set may include determining one or more or all of the possible combinations of identity values which could have given rise to the test sample analysis data set. The processing of the test sample analysis data set may include determining one or more or all of the possible combinations of allele values which could have given rise to the test sample analysis data set. One or more or all of the identity values and/or combinations thereof may be provided with a further data element. The further data element may indicate one of the following:

an identity value, such as an allele, which is accepted as being an identity value, such as an allele;

an identity value, such as an allele, which is not accepted as an identity value, such as an allele, for instance because it is a not identity variables, such as a non-allelic source;

an identity value, such as an allele, which may or may not be an identity value, such as an allele.

Two or more different further data elements may be used to indicate different positions. The further data element and/or the selected further data element from the possible data elements may be added to the identity value and/or the value of the identity value. For instance, the test sample results data set may include the value or size of designation of the allele and a further data element.

The use of further data elements may be applied in respect of the test sample results data set for one or more or all of the loci considered.

The further data element may not be used in the search term and/or may not be used in the comparing of the test sample and/or may not be used in the determination of potential matches.

The probability and/or probabilistic weighting give to a potential match may be weighted. The weighting may be influenced by the presence of a further data element and/or may be set by the presence of a further data element. The weighting may be influenced by the particular further data element present and/or may be set by the presence of particular further data element. The presence of a further data element and/or the particular data element present may influence the position of the potential match in a list of potential matches, particularly when ranked according to their probability and/or probabilistic weighting.

The probability and/or probabilistic weighting give to a potential match may be weighted. The weighting may be influenced by whether the potential match involves identity value, such as allelic sources and/or whether the potential match involves non identity values, such as non-allelic sources and/or involves suspected non-identity values, such as non-allelic sources.

A higher weighting may be given to a potential match which involves an identity value, such as allelic sources compared with a potential match which involves a non-identity values, such as non-allelic sources. A higher weighting may be given to a potential match which involves an identity value, such as allelic sources compared with a potential match which involves suspected non-identity values, such as non-allelic sources. A higher weighting may be given to a potential match which involves suspected non-identity values, such as non-allelic sources compared with a potential match which involves a non-identity values, such as non-allelic sources.

A higher weighting may be given to a potential match which involves an identity value, such as allelic sources, for both the test sample and the another sample compared with a potential match which involves a non-identity values, such as non-allelic sources, for one or both of the test sample or the another sample. A higher weighting may be given to a potential match which involves an identity value, such as allelic sources compared with a potential match which involves suspected non-identity values, such as non-allelic sources, for one or both of the test sample or the another sample. A higher weighting may be given to a potential match which involves suspected non-identity values, such as non-allelic sources for one or both of the test sample or the another sample compared with a potential match which involves a non-identity values, such as non-allelic sources for one or both of the test sample or the another sample.

The defining of the search term may involve providing one or more search terms. The defining of the search terms may involve providing one or more search terms to include one or more and preferably all of the test sample results in the test sample result data set. One search term may include one or more or all of the test sample results. Preferably the number of search terms is less than the number of test sample results in the test sample data set. A search term may include one or two identity values. One or both of the identity values may be a numerical value for the identity value. One or both of the identity values may be a representation of a single identity value, such as an allele size or designation. One or both of the identity values may be a non-numerical value for the identity value. Preferably at least one, and ideally all, of the non-numerical values are not constrained identity values. Preferably at least one, and ideally all of the non-numerical values are unconstrained identity values.

The search term may include one or two or three or more search terms for each identity variable, such as a locus.

The replacement of may consider a first identity value and the provision of a search term which equals and/or includes that first identity value. The replacement may consider a second identity value against the search term. The search term may be unaltered if the search term equals and/or includes the second identity value. The search term may be modified if the search term does not equal or include the second search term, for instance by changing a value in the search term to an unconstrained identity value. The search term may have a further search term added to it where the search term does not equal or include the second search term and changing a value in the search term to an unconstrained identity value would not make the search term equal or include the second search term. The replacement may include one or more further identity values being considered against the then present form of the search term. This may result in the present form of the search term being unaltered and/or modified by changing a value in the search term to an unconstrained identity value and/or have a further search term added to it.

One or more and preferably all of the constrained identity values are replaced.

The unconstrained identity value may have a value equal to all of the possible identity values, for instance all the allele values for that locus. The possible identity values may be those which exist or which are known for that identity variable, for instance that locus. The possible identity values may include the value “unknown” for the identity variable. The possible identity values may not be deemed to include the value “unknown” for the identity variable.

The obtaining of the another sample may be provided by accessing a record in a database. The record may contain the another sample and/or another sample result data set and/or another sample result or results.

The another sample and/or another sample result data set and/or another sample result or results may have any of the features, options or possibilities provided for the test sample and/or test sample result data set and/or test sample result or results.

The identity values for the another sample result may have any of the features, options or possibilities provided for the identity values for the test sample.

The comparing step may be provided by a computer and/or computer software and/or logic provided for comparing test samples which lack an associated probability and/or probabilistic weighting.

The comparing step may include another sample results having an associated probability and/or probabilistic weighting and/or another sample results not having an associated probability and/or probabilistic weighting.

The comparing of the another sample result with the search term may consider whether the another sample result equals or is within the search term, for instance by being equal to or within one of more of the parts forming the search term.

The complying with the requirements of the search term may be acknowledged where the another sample result equals or is within the search term, for instance by being equal to or within one of more of the parts forming the search term.

The not complying with the requirements of the search term may be acknowledged where the another sample result does not equals or is not within the search term, for instance by being not equal to or not within all of the one of more of the parts forming the search term.

A potential match may be a match between a test sample result and an another sample result for which the requirements of the search term are complied with.

Where another sample result does not comply with respect to any one of the different identity variable being compared in the search term, then a non-match occurs.

Where another sample result complies with respect to the search term, the another sample result may be further compared with the test result. This further comparison may be in terms of the identity value of the one or more identity variables considered, rather than the search terms that represents them. This may be provided in a further comparison and/or by the allocation of the probability and/or probabilistic weighting to that another sample result according to the identity value of the one or more identity variables of the another sample result. Where the another sample result has identity value for one or more of the identity variables which are not in the test result identity values, then a low or zero probability and/or probability weighting may be allocated.

The allocating of a probability and/or a probabilistic weighting may be made based upon a value associated with a test result. The value may be associates with the test result during the processing of the test sample analysis data set to the test sample results data set. The value may be associated with the test result according to the approach detailed in International Patent Publication number WOWO2009/066067 and/or US Patent Application Publication number US2009/0132173 and/or International Patent Publication number WO2010/116158.

The probability and/or probabilistic weighting allocated for one identity variable may be combined with the probability and/or probabilistic weighting allocated to one or more or all the other identity variables. They may be combined by addition or multiplication.

The ranking be from highest value to lowest value. The ranking may be from the lowest value to the highest value. Those rankings above a threshold value may be excluded and/or included in a further consideration. Those rankings below a threshold value may be excluded and/or included in a further consideration.

The combined value may be used in the ranking.

The further consideration may be in legal proceedings as evidence and/or in determining an action to take in an investigation by a law enforcement authority.

The method may be applied to test sample analysis data sets and/or test sample results data sets and/or test results and/or another sample analysis data sets and/or another sample results data sets and/or another results with respect to a plurality of different identity variables, for instance a plurality of different loci.

The identity values for one or more of the identity variables considered in the analysis may be obtained for a plurality of identity variables, preferably 5 or more, ideally with the identity variables being different loci.

The search term defined may include one or more search terms, with one or more search terms being obtained to cover the identity values for a plurality of identity variables, preferably 5 or more, ideally with the identity variables being different loci.

The comparing may be provided in respect of the search term and/or the another sample for a plurality of identity variables, preferably 5 or more, ideally with the identity variables being different loci.

The complying with the requirements of the search term to provide a potential match may require complying for one or more or all of a plurality of identity variables, preferably 5 or more, ideally with the identity variables being different loci.

Any of the proceeding aspects of the invention may include the following features, options or possibilities or those set out elsewhere in this document.

Various embodiments of the invention will now be described, by way of example only and with reference to the accompanying drawings in which:

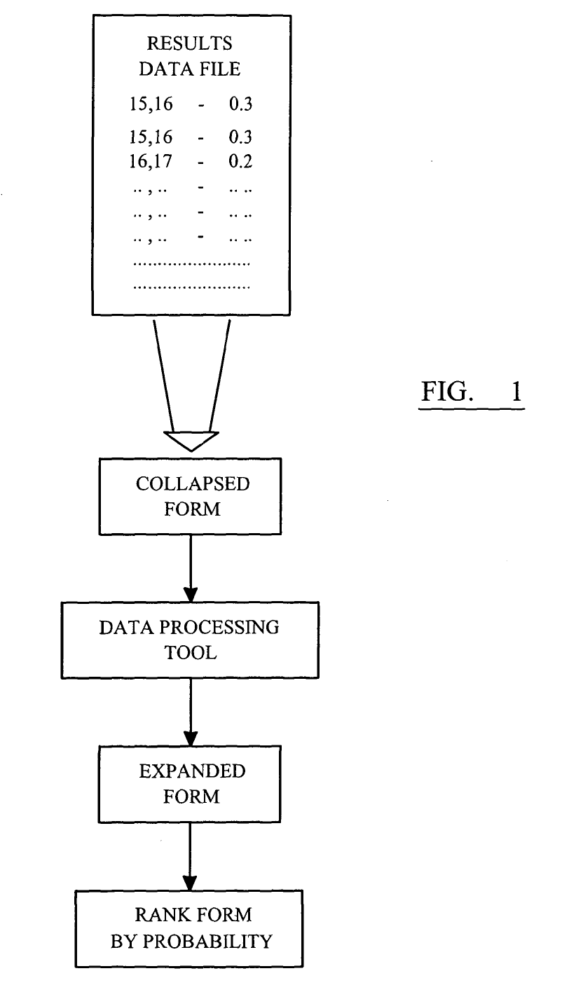

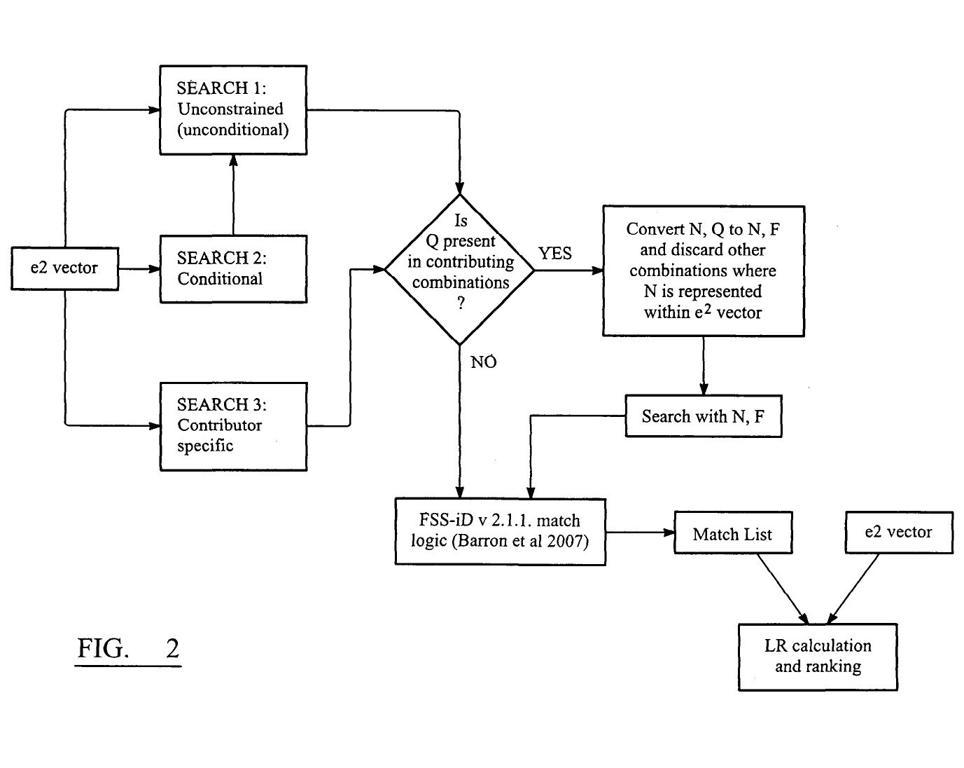

FIG. 1 is a schematic illustration of a part of the processing system according to an embodiment of the present invention;

FIG. 2 is a processing system flow diagram for a part of the processing system according to a further embodiment of the present invention;

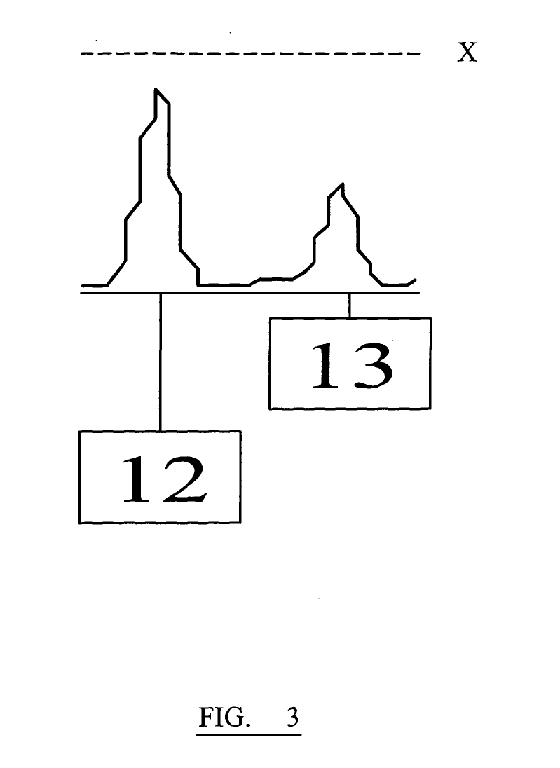

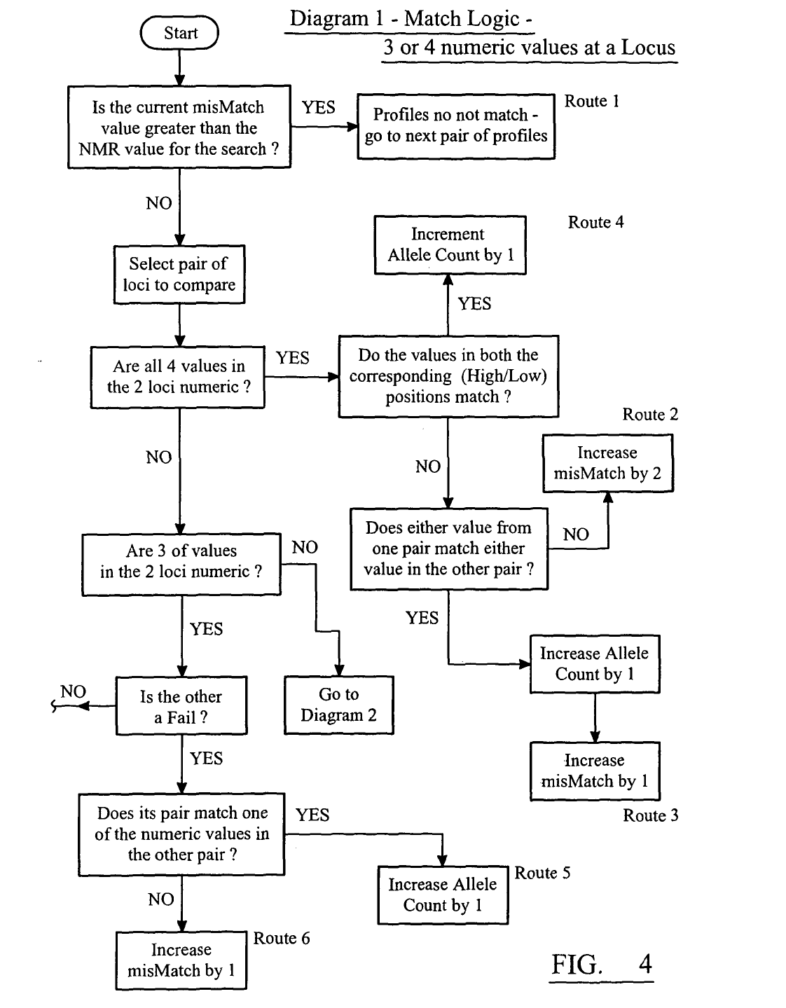

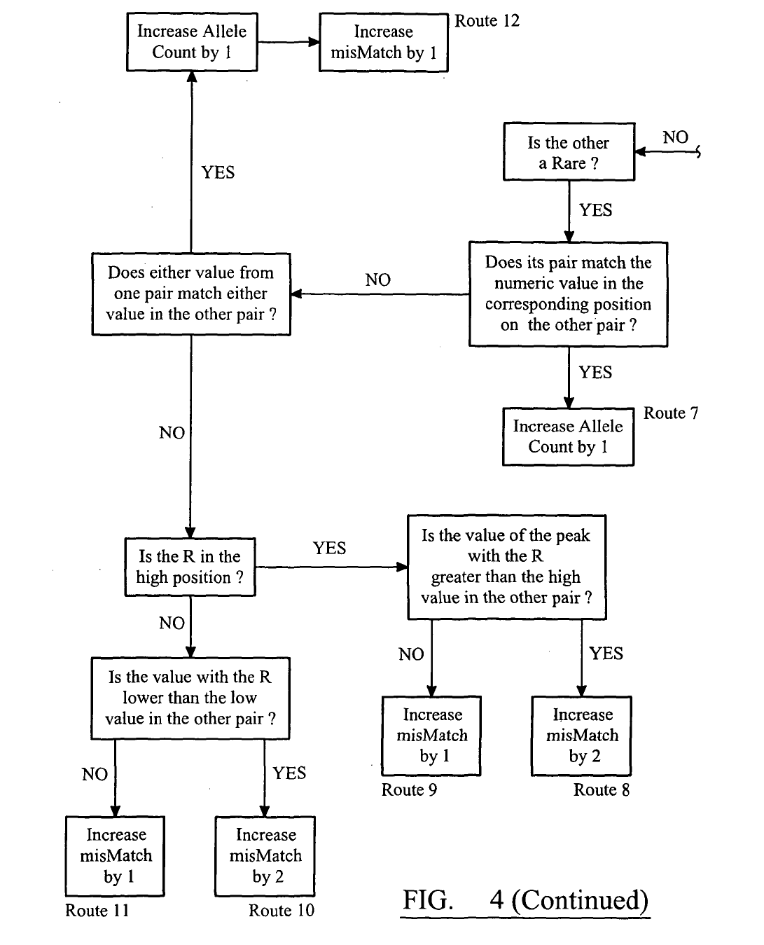

FIG. 3 is a schematic illustration of a detected signal for a locus;

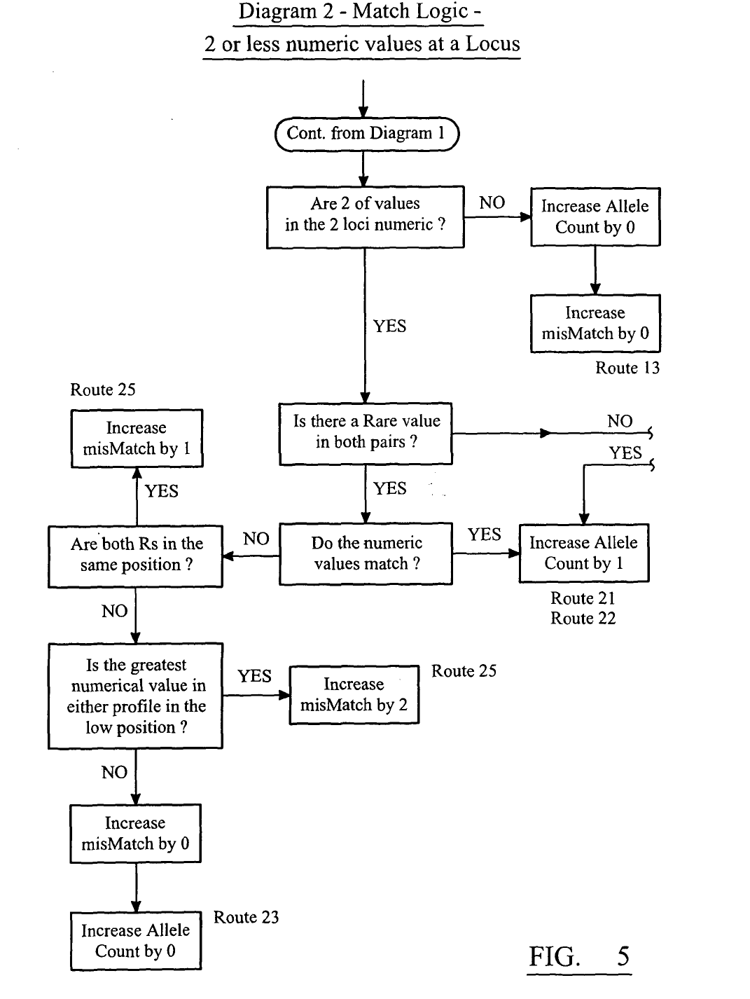

FIG. 4 is a illustration of a matching logic suitable for use in the present invention and useful where three or four numerical values within a locus arise; and

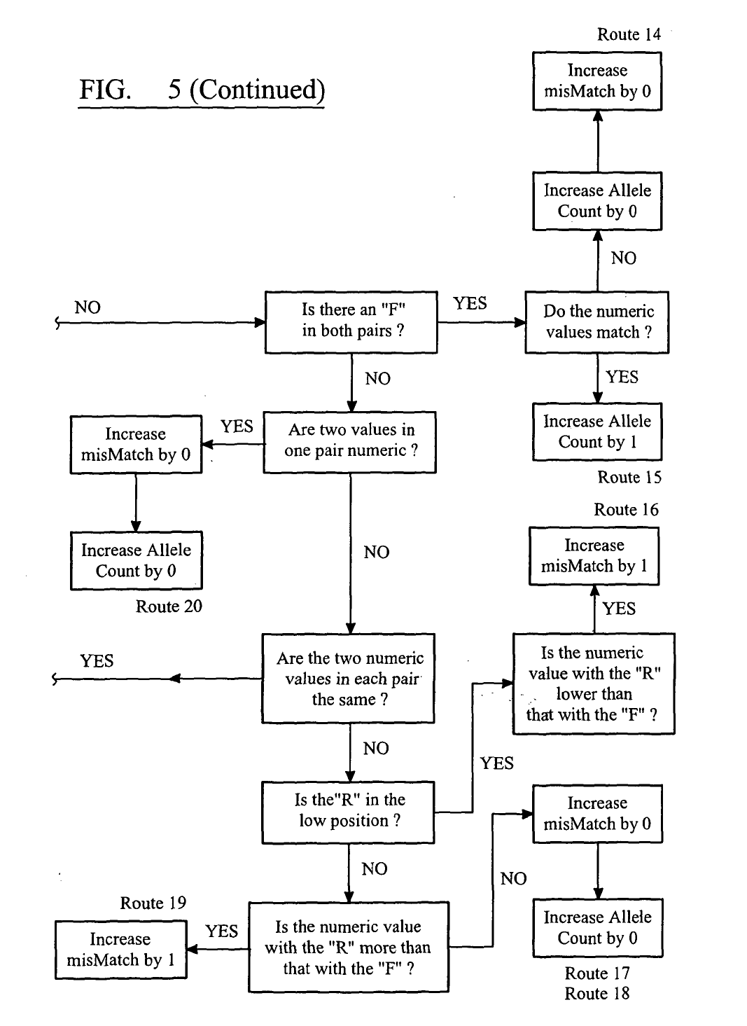

FIG. 5 is a illustration of a matching logic suitable for use in the present invention and useful where two or less numerical values within a locus arise.

### CONTEXT AND BACKGROUND TO THE INVENTION

The present invention is concerned with improving the interpretation of results from DNA analysis and the manner in which the results therefrom are considered against other results to give a match or a likelihood of a match.

Basically, the DNA analysis involves taking a sample of DNA and analysing the variations present at a number of loci. The identities of the variations give rise to a data set which is then interpreted to give a profile or genotype. The extent of interpretation required can be extensive and/or introduce uncertainties. This is particularly so where the DNA sample contains DNA from more than one person, a mixture.

There is often a need to consider various hypotheses for the identities of the persons responsible for the DNA and evaluate the likelihood of those hypotheses, evidential uses.

There is often a need to consider the analysis profile or genotype against a database of profiles or genotypes, so as to establish a list of stored profiles or genotypes that are likely matches with the analysis profile or genotype, intelligence uses.

In support of this analysis, the applicant has developed and disclosed a mathematical specification of a model for computing likelihood ratios (LRs) that uses peak heights taken from such DNA analysis. The approach draws on an estimation of a two-dimensional, 2D, probability density function, pdf, which is estimated from the heights or areas of peaks observed after the analysis of control samples. Such pdf's may be generated from heterozygous donors and separately from homozygous donors. The approach goes on to calculate the probability of dropout and achieve other benefits. Full details of these developments are to be found in International Patent Publication number WOWO2009/066067 and/or US Patent Application Publication number US2009/0132173, the contents of both of which are fully incorporated herein by reference, particularly with respect to the analysis of the samples, their mathematical expression and their comparison with others, including the determination of the likelihood ratio for a match between them.

Subsequently, the applicant has developed that technology further. The statistical model now provides for computing likelihood ratios for single profiles and mixed profiles while considering peak heights or areas, but also takes into consideration allelic dropout and stutters. In this way, the technique makes far greater use of a far greater proportion of the information in the results and hence give a more informative and useful overall result.

To achieve this, the present invention includes the use of a number of components. The main components of the approach are:

1. An estimated PDF for homozygote peaks conditional on DNA quantity;

2. An estimated PDF for stutter heights conditional on the height of the parent allele;

3. An estimated joint probability density function (PDF) of peak height pairs conditional on DNA quantity;

4. A latent variable X representing DNA quantity that models the variability of peak heights across the profile.

5. The calculation of the LR is done separately for the numerator and the denominator. The overall joint PDF for the numerator and the denominator can be represented with Bayesian networks (BNs).

Full details of these further developments to the technology are included in International Patent Publication number WO2010/116158, the contents of which are fully incorporated herein by reference, particularly with respect to the analysis of the samples, their mathematical expression and their comparison with others, including the determination of the likelihood ratio for a match between them.

The use of such technology, and potentially other approaches, for the consideration of the DNA sample gives a results data set. This results data set includes a far larger volume of data in the results data set than was produced under previous approaches. This is beneficial in terms of the information which may be obtained and the ability to consider a wider range of possible matches. The volume of data in the results set may be larger because instead of reaching a single or relatively limited number of possibilities (expressed as possible alleles at one or more loci, through to expression as a profiles or genotypes through interpretation of the results), the results include a far larger number of possibilities (expressed as possible alleles at one or more loci, through to expression as a profiles or genotypes). However, the volume and/or form of the results data set also creates problems with respect to the computation resources and/or time needed to process those results in the subsequent data processing stages. A much larger number of possible profiles need to be considered against other profiles to see if there is a match.

### Overview of the Invention

Within the results data set, one of the key reasons for a larger volume of data is that one or more of the variables have a value which is context specific. For instance, the data set may consider allele dropout in a constrained manner. Thus presuming the result is stated as showing allele 15, and an unobserved allele (for instance one believed to have dropped out), then this is represented as a result 15, Q. The result 15, Q does not mean allele 15 and any other unobserved allele. Rather it means a constrained set depending on the circumstances of that result. Thus, were the result is constrained and is defined to be one of: allele 15 and allele 16; allele 15 and allele 17; allele 15 and allele 18, the values represented by 15, Q are 15,16; 15, 17; 15,18. The meaning of Q is result/context specific. Between results, Q can mean different things. In effect, Q is anything other than what is observed in that context; so if 16, 17, 18 are observed then Q is anything other than 16, 17, 18. If the search logic is provided in a form which can accommodate this, then that logic becomes enormously expanded and complex.

To counteract this position, the invention takes the results data set and collapses it down. The resulting volume of data and resulting form of the data within the manipulated results data set is then suitable for processing according to one or more existing data processing tools, for instance FSSiD DNA analysis software published by Forensic Science Service Limited. The form and operation of the compression manipulation, which provides the interface, is important to the success of the present invention.

Rather than having to consider all of these, the manipulated results data set considers 15,Q as being 15,F, where F is any allele. This is easier to process as F has the same meaning in each result. The logic is thus maintained in a simpler form and does not need expansion.

After data processing, the resulting processed results data set can have the manipulation reversed. This expands the results and gives an expanded results data set, potentially providing to an ordered list of data. The probability information which is also obtained can then be used to rank that ordered results data set into a probability ranked results data set.

The overall process is shown schematically in the stages of FIG. 1.

Having made this simplification, the matching logic in the data processing tool can operate as designed. This means that the old data processing tool is able to successfully consider and process results data sets generated in the new form described, even where that new form cannot be processed directly.

### DETAILED DESCRIPTION

In a more detailed embodiment, the invention is uses the technology described in the applicant's previous patent applications (referenced above) to analyse and express the DNA sample content in a form suitable for comparison.

Starting from this results data set, in the applicant's technology form, e² DNA analysis software, for instance, the processing system advances according to the flow diagram of FIG. 2.

In the first step, a decision is made by the operator as to which search type to operate. There are three possibilities in this embodiment. Search type 1 is an unconstrained or unconditional search; search type 2 is a conditional search; and search type 3 is a contributor specific search.

Having made this decision, in the next stage, the process system considers whether or not any of the combinations in the results data set have the Q form present. If that form isn't present, then the collapsing of the results data set is not needed and the processing can pass on to the data processing tool using the matching logic. If the Q form is present, the collapsing of the results data set is needed.

The Q form is often present where the low level of DNA in the DNA sample gives rise to an unobserved allele being a possibility. In effect, not all the present alleles are observed. This is seen as occurring when the signal level for the observed alleles is below a given threshold. Where the signal level for the observed alleles is above that given threshold, the observed alleles are equated to being all the present alleles for the DNA sample.

The combinations featuring a variable in the Q form are converted to combinations featuring a variable in the F form.

Considering the example shown in FIG. 3, signals are observed for both allele size 12 and allele size 13. However, the observed alleles have signal levels which are below the threshold X.

This profile could arise with a number of different allele combinations, as set out below in Table 1.

TABLE 1

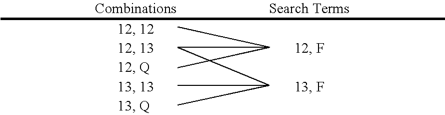

In the first left-hand group of combinations, 12,12 represents a homogeneous situation, the combination 12,13 a heterogenous situation and the combination 12,Q a situation where the second allele is an allele other than an allele excluded by the constrain, in this case, any allele other than 12 or 13. In the second left-hand group of combinations, the homogenous situation, 13,13 is a possibility and the 13,Q situation is another. In this case, Q has a value as being any allele other than 12 or 13.

To reduce the complexity of the searching and matching logic needed, the present invention provides a Q to F conversion. When Q is converted to an F, the total number of search terms is reduced, right-hand search terms, such that all of the possible combinations are considered by the numerical (N), F search term. This is because F can be any allele within the analysed locus.

Depending upon the search option selected, the impact of the manipulation may vary.

In the illustrations that follow, a 2-person mixture with three alleles present and observed at a locus, 14,15,16 is considered in relation to a search of each search type. Table 2 lists the possible combinations that would be possible, for instance as generated by the e² mathematical model, with respect to the alleles for contributor 1 and the alleles for contributor 2. In practice, each pair combination is also associated with a probability value, but for the purposes of this illustration no values have been presented in column Pr of Table 2.

TABLE 2

| | Search 1 | Search 2 | Pr |
|---|---|---|---|
| Combination 1 | 14, 14 | 15, 16 | . . . |
| Combination 2 | 14, Q | 15, 16 | . . . |
| Combination 3 | 15, 15 | 14, 16 | . . . |
| Combination 4 | 15, Q | 14, 16 | . . . |
| Combination 5 | 16, 16 | 14, 15 | . . . |
| Combination 6 | 16, Q | 14, 15 | . . . |
| Combination 7 | 14, 15 | 14, 16 | . . . |
| Combination 8 | 14, 15 | 15, 16 | . . . |
| Combination 9 | 14, 15 | 16, 16 | . . . |
| Combination 10 | 14, 15 | 16, Q | . . . |
| Combination 11 | 14, 16 | 15, 15 | . . . |
| Combination 12 | 14, 16 | 14, 15 | . . . |
| Combination 13 | 14, 16 | 15, 16 | . . . |
| Combination 14 | 14, 16 | 15, Q | . . . |
| Combination 15 | 15, 16 | 14, 14 | . . . |
| Combination 16 | 15, 16 | 14, 15 | . . . |
| Combination 17 | 15, 16 | 14, 16 | . . . |
| Combination 18 | 15, 16 | 14, Q | . . . |

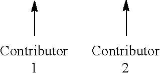

Given this set of combinations, each of the three search types will now be illustrated.

### Search Type 1

For search type 1, the unconstrained or unconditional search type, no combinations are excluded in an initial step. All the combinations remain, but then the manipulation consolidates the combinations across contributors and thus reduces the search list size. For example, if 14,Q is converted to 14,F as described above, then the combinations 14, 14; 14; Q; 14,15; 14,16 in Table 2 above would simplify to the combination 14,F. This is because the F wildcard will include the 14, 15 and 16 alleles during the searching and matching process. If the manipulation is repeated for the 15 and 16 allele then this will result in search terms 15, F and 16, F and the full simplification shown in Table 3 below for searching.

TABLE 3

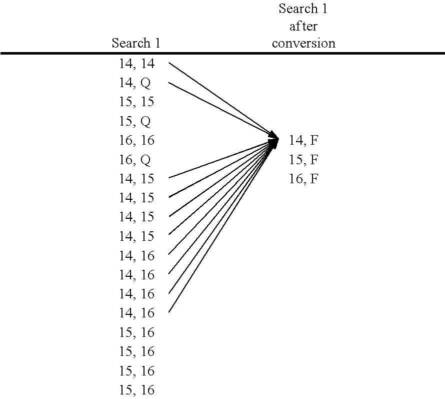

### Search Type 2

For search type 2, the conditioned search, an initial step removes combinations that do not match the conditioning profile. Then the manipulation consolidates the combinations across contributors.

Referring back to the starting point of Table 2 and deciding to condition on 14,15 in the initial step, then this means that the e² vector is revised to a possible list as summarised in Table 4 below, where values of 14,15 are removed from the vector.

After this conditioning, the search terms for each contributor are then manipulated in the same manner as for search type 1. Hence, as Q is present in a combination for contributor 1, that is covered by 14,F and 15,F. For contributor 2, the combinations simplify to become 14,F and 15,F too.

TABLE 4

| | Search 1 | Search 2 | Pr |
|---|---|---|---|
| Combination 1 | 14, 14 | 15, 16 | . . . |
| Combination 2 | 14, Q | 15, 16 | . . . |
| Combination 3 | 15, 15 | 14, 16 | . . . |
| Combination 4 | 15, Q | 14, 16 | . . . |
| Combination 11 | 14, 16 | 15, 15 | . . . |
| Combination 13 | 14, 16 | 15, 16 | . . . |
| Combination 14 | 14, 16 | 15, Q | . . . |
| Combination 15 | 15, 16 | 14, 14 | . . . |
| Combination 17 | 15, 16 | 14, 16 | . . . |
| Combination 18 | 15, 16 | 14, Q | . . . |

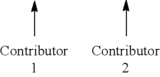

### Search Type 3

For search type 3, again the starting point is the combinations of Table 2. In this case, the contributor specific search, the initial step consolidates combinations within contributors. For example, if a user has observed 14, 15 and 16 alleles and is considering only allele 16 as the search term, then based on Table 2 above the e² vector would be revised to that shown in Table 5 below. Combinations without 16 are omitted.

Once again, the manipulation can then be performed to leave search 16,F for contributor 1 and search 16,F for contributor 2.

TABLE 5

| | Search 1 | Search 2 | Pr |
|---|---|---|---|
| Combination 1 | | 15, 16 | . . . |
| Combination 2 | | 15, 16 | . . . |
| Combination 3 | | 14, 16 | . . . |
| Combination 4 | | 14, 16 | . . . |
| Combination 5 | 16, 16 | | . . . |
| Combination 6 | 16, Q | | . . . |
| Combination 7 | | 14, 16 | . . . |
| Combination 8 | | 15, 16 | . . . |
| Combination 9 | | 16, 16 | . . . |
| Combination 10 | | 16, Q | . . . |
| Combination 11 | 14, 16 | | . . . |
| Combination 12 | 14, 16 | | . . . |
| Combination 13 | 14, 16 | 15, 16 | . . . |
| Combination 14 | 14, 16 | | . . . |
| Combination 15 | 15, 16 | | . . . |
| Combination 16 | 15, 16 | | . . . |
| Combination 17 | 15, 16 | 14, 16 | . . . |
| Combination 18 | 15, 16 | | . . . |

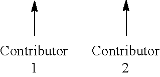

### Matching Processing

Having performed the manipulation to get the search terms, the process system applies the matching logic by a data processing tool.

Because of the manipulation of the results data set to a form acceptable to a wide range of data processing tools, this allows the use of existing data processing tools, such as Forensic Science Service's FSSiDs matching logic to be used. Such systems incorporate the numerical (N), F approach to data. This avoids the need to develop and then validate a new data processing tool with the time and expense issues that arise therefrom.

The same data processing tool can be used for processing data of the new type which includes Q form combinations, whilst still allowing processing of the older type data lacking in Q form combinations. The flexibility of the processing system is thus increased.

The form of the matching logic used can be varied and updated without detracting from the present invention. By way of illustration, a matching logic for use for three or more numerical values within a locus is shown in FIG. 4 and for two or less numerical values in FIG. 5.

The data processing tool compares the search terms with an individual stored genotype or profile for a match. The matching process is repeated for the very large number of genotypes or profiles that are stored in the database. The whole of the database, or a part thereof according to one or more constraints, may be considered. Use of the data processing tool produces possible matches against the existing data, generally stored in a database, such as The National DNA Database operated in the UK. The searching and matching is conducted for each of the loci to produce a match list.

In basic terms, the match list is formed as follows. If the search term for a locus is 16,F and the stored genotype or profile is 15,16 for that locus, then there is a match for that locus. If the stored genotype or profile is 13,14 for that locus, then there is no match for that locus with the 16,F search term. Of course, the process is performed across multiple loci and so matches may not occur at other loci, even where there is a match at one or more of the loci.

Expanding the demonstration of the principle to a 5 locus hypothetical case, the search terms are defined in the second row of Table 6, with five stored genotypes or profiles represented in the other rows. In this case, there are four instances of the stored profile or genotype matching with that set of search terms. In the other example, the last row, there is no match at locus 2 and 4, even thought the others match; overall this is a non-match.

TABLE 6

| | Locus 1 | Locus 2 | Locus 3 | Locus 4 | Locus 5 |
|---|---|---|---|---|---|
| Search Term | 14F, 15F | 8F, 9F | 16F, 18F | 14, 15 | 22F, 24 |
| Example no. 1 | 14, 16 | 9, 11 | 16, 18 | 14, 14 | 22, 24 |
| Example no. 2 | 15, 16 | 9, 9 | 18, 20 | 14, 14 | 22, 26 |
| Example no. 3 | 14, 14 | 8, 10 | 16, 18 | 14, 15 | 24, 26 |
| Example no. 4 | 14, 17 | 8, 9 | 16, 18 | 15, 15 | 24, 26 |
| Example no. 5 | 15, 15 | 10, 11 | 16, 20 | 12, 13 | 22, 22 |

Having represented the Table 6, the second part of the data processing is represented in Table 7. In practice, both parts are conducted in a single set of process steps. In Table 7, the probability of each match is brought into consideration.

In Table 6, Example no. 5 is a non-match for locus 2 and locus 4 and hence has a zero probability overall due to the non-matches in Table 7.

In the case of Example no. 1 and Example no. 2, the stored genotypes or profiles have been obtained using the new approach referenced above which gives rise to a probability weighting being stored in the database as part of the result. These can then be used to populate the probability values for the various loci and hence a total probability value. The probability values (illustrative only) for each of the loci are multiplied together to give the total probability value. Addition or other weightings can be given to the combination of the probabilities.

In the case of Example no. 3, the stored profile or genotype from the database does not have a probabilistic weighting stored with it, for instance because it is data that predates the implementation of the new approach mentioned above. As a result, a different approach is taken to generate the probability value and hence that matches position in the ranked list. In such a case, it is possible to assign a weighting which is obtained from a model, for instance reflecting the probability of that allele or pair of alleles occurring in the population or a sub-set thereof, potentially in combination with one or more adjustment factors, for instance to bring the magnitude of the probability into line with the probabilities where the weighting has been stored.

In the case of Example no 4, the match only arose because of the use of the F search term, rather than the context specific constrained search term Q. Thus for locus 1, the considered search was 14,F or 15,F and this matched with Example no. 4 which has 14,17. However, working back from 14,F, 15,F to expand back to the original list of combinations, that set of combinations included a constrained value for Q, where Q was 14, 15 or 16. On that basis, neither 14,Q or 15,Q matches with the observed result, 14,17 and so that possible match is excluded. The correct probability is zero, despite the match at the 14,F level. If Q was not constrained in this case, but was any observed allele, then 14,17 would match 14,Q since allele 17 in 14,17 is unobserved.

TABLE 7

| | Prob | | Prob | | Prob | |
|---|---|---|---|---|---|---|
| Ex | 1 | Prob 2 | 3 | Prob 4 | 5 | Total Prob |
| Example no. 1 | 0.017 | 0.022 | 0.017 | 0.023 | 0.009 | 1.316 × 10⁻⁹ |
| Example no. 2 | 0.008 | 0.018 | 0.009 | 0.023 | 0.011 | 3.279 × 10⁻¹⁰ |
| Example no. 3 | 0.011 | 0.009 | 0.017 | 0.015 | 0.014 | 3.534 × 10⁻¹⁰ |
| Example no. 4 | 0 | 0.015 | 0.017 | 0.015 | 0.014 | 0 |
| Example no. 5 | 0.018 | 0 | 0.015 | 0 | 0.023 | 0 |

Of course, the above approach combines the position for as many loci (Table 6 columns) as are desired and considers the position in respect of a very large number of different profiles or genotypes (Table 7 columns).

Where there are no matching alleles for a locus, then the match is assigned a weight (or probability) of zero. The occurrence of a locus with no matching alleles may result in the comparison process stopping or it may be completed across the other loci.

In effect, the match list has been amalgamated and linked back to the original e² vector produced by the model used to obtain the probabilities. This will then assist in the generation of the required Likelihood Ratio (LR) values for a match.

The total probability value can be used to give a value for the likelihood of a match between the sample being analysed and the stored genotype or profile. The differences in values can be used to rank the results.

If there is a need to reduce the number of combinations in the match list, then it is possible to impose a probability cut-off level for that list. In effect, where combinations are the wrong side of a set probability level, they are eliminated from the search list going forward.

Overall, by collapsing, data processing and then expanding the terms in the manner described above, the data processing logic remains backward compatible with legacy data. Hence, the old data (lacking the probabilistic weighting) and the new data (having the probabilistic weighting) can both be considered in the same searches and/or using already validated and established matching logic. This is a significant advantage.

The results of the process can be used to determine one or more of many possible subsequent actions. In particular, the results could cause a subsequent sample to be collected and/or analysed to determine the alleles or allele identities it possesses, or other information. The analysis will generally include the same or similar collection, sample preparation, sample purification, PCR amplification or other amplification approach, sized based separation of the products (such as electrophoresis) and detection of the results (for instance by detection of fluorescence) steps as are used to initially analyse the sample. The analysis may relate to a subsequent sample from the same source as the test sample and/or from the same source as the reference sample on the database. These collection steps and analysis steps are a direct result of the information revealed by the original analysis and comparison process.

### Overview of a Further Development

Within the results data set above, the two types of data present are either alleles of stated sizes or alleles of a non-stated size. The alleles of a stated size may, for instance be, allele 15, allele 16. Where the alleles of a non-stated size are referred to, then these may be designated Q (according to the old approach, where the meaning of Q is constrained and/or is result and context specific, for instance 15 or 16 or 17); or may be designated F (according to the approach of the invention, where the meaning of F is any allele).

Thus the results data set is an expression of alleles.

However, to reach the position where a meaning as an allele is given, there is a need for interpretation. That interpretation may exclude peaks which are not taken to be definite alleles. That interpretation may exclude peaks which could be an allele, but may not be an allele, and/or peaks which are very unlikely to be alleles. Such peaks may be due to stutters or contamination or artifacts etc. They can be generally be thought of as being non-allelic.

In this revised form of the invention, the manner in which the results data set is formed and is expressed is changed. Instead of excluding non-allelic peaks and/or ambiguous peaks, these may be included in the results data set.

The size, that is the allele position, of the peak is recorded. The decision on whether that peak is allelic or non-allelic is also recorded.

For instance, peaks may be observed, where Peak A is an allele peak and has size 16 and Peak B is a potential allele peak, but also may be caused by stutter. The size of Peak B is also noted, with a value of 15 to reflect its position in the same way. Because Peak A is called an allele peak in the results, the designation A16 is applied. Because Peak B is not a definite allele in the results (although it may be an allelic peak), the designation NA15 is applied. Thus the result becomes NA15,A16 for that locus.

An equivalent approach is applied across the other loci being considered.

Such an approach may expand the number of terms being fed to a search, but more frequently will increase the number of results a search generates.

Beneficially, however, the matching logic in the data processing tool can operate as designed. Matches between the sizes are looked for. The designation as allele, A, or non-allele, NA, is not significant in the matching logic and is not used there. This means that the old data processing tool is able to successfully consider and process results data sets generated in the new form described, even where that new form cannot be processed directly.

After data processing, the resulting processed results data set can have the manipulation reversed. This expands the results and gives an expanded results data set, potentially providing to an ordered list of data. Thus the results data set is matched to a series of other others, a potential match list. The probability information which is also obtained can then be used to rank that ordered results data set into a probability ranked results data set.

The probability information and/or ranking may reflect the extent to which the potential match is based upon an allelic to allelic designated matches at one or more of the allele sizes and/or is based upon allelic to non-allelic designated matches at one or more of the allele sizes and/or is based upon non-allelic to non-allelic designated matches at one or more of the allele sizes. A higher probability and/or ranking may be given to a match of allelic to allelic designations than a match based upon non-allelic to non-allelic designations.

Thus, the above mentioned example of NA15, A16 may match with four different results as a consequence of the matching process. These may be as shown in Table 8.

TABLE 8

| Match Number | Results Data Set | Other Data Set |
|---|---|---|
| 1 | NA15, A16 | A15, A16 |
| 2 | NA15, A16 | A15, NA16 |
| 3 | NA15, A16 | NA15, NA16 |
| 4 | NA15, A16 | NA15, A16 |

In the next operation, the significance of the allelic or non-allelic designation is taken into account through a weighting. This gives the revised position of a ranked list; see Table 9.

TABLE 9

| Match Number | Results Data Set | Other Data Set | Weight | Rank |
|---|---|---|---|---|
| 1 | NA15, A16 | A15, A16 | 0.8 | 1 |
| 3 | NA15, A16 | NA15, A16 | 0.6 | 2 |
| 4 | NA15, A16 | A15, NA16 | 0.4 | 3 |
| 2 | NA15, A16 | NA15, NA16 | 0.2 | 4 |

The above approach can be used in cases where the meaning of Q is result/context specific and/or where F has the same meaning in each result, any allele. Thus that form of designation can be used alongside the allelic/non-allelic designations.

1. A method of analysing and comparing a test sample with another sample, wherein:
the test sample is analysed, the analysis producing test sample analysis data set;
the test sample analysis data set is processed to give a test sample results data set;
defining a search term relating to the test sample results data set;
obtaining the another sample to compare with the test sample;
comparing the another sample result with the search term to inform on the another sample being a potential match with the test sample.

2. A method according to claim 1 in which where one or more potential matches are
identified the method further includes performing an analysis of a subsequent sample.

3. A method according to claim 2 in which the analysis of the test sample includes one or
more of sample preparation, purification, amplification, size based separation or electrophoresis and/or the analysis of the subsequent sample includes one or more of sample preparation, purification, amplification, size based separation, electrophoresis, fluorescence or light detection.

4. A method according to claim 2 in which the subsequent sample is a subsequent sample taken from the test sample and/or a subsequent sample taken from the same source as the test sample.

5. A method according to claim 1 wherein:
the test sample is analysed, the analysis producing test sample analysis data set;
the test sample analysis data set is processed by a computer implemented step to give a test sample results data set, the test sample results data set including one or more test sample results, a test sample result including an identity value for one or more of the identity variables considered in the analysis, the possible identity values including a constrained identity value, the constrained identity value being two or more of the possible identity values and less than all of the possible identity values;
defining, in a computer implemented step, a search term from one or more of the test sample results, the defining of the search term involving the replacement of one or more of the constrained identity values with an unconstrained identity value, the unconstrained identity value being all the possible identity values;
obtaining, in a computer implemented step, the another sample to compare with the test sample, the another sample being in the form of an another sample result data set, the another sample result data set including one or more another sample results, an another sample result including an identity value for one or more of the identity variable considered in an analysis of that another sample;
comparing, in a computer implemented step, the another sample result with the search term, the another sample result complying with the requirements of the search term and thereby providing a potential match between the another sample and the test sample or the another sample result not complying with the requirements of the search term and thereby not providing a potential match between the another sample and the test sample;
allocating, in a computer implemented step, a probability and/or a probabilistic weighting to one or more of the potential matches;
ranking, in a computer implemented step, the one or more potential matches provided with a probability and/or a probabilistic weighting according to the value of the allocated probability and/or probabilistic weighing.

6. A method according to claim 5 in which the constrained identity value has a value equal to two or more of the possible identity values, but less than all the possible identity values.

7. A method according to claim 5 in which the unconstrained identity value has a value equal to all of the possible identity values.

8. A method according to claim 5 in which the defining of the search term may involve providing one or more search terms, a search term including one or two identity values, where in one or both of the identity values may be a numerical value for the identity value and/or one or both of the identity values may be a non-numerical value for the identity value.

9. A method according to claim 8 in which all of the non-numerical values are unconstrained identity values.

10. A method according to claim 1 in which the obtaining of the another sample is provided by accessing a record in a database.

11. A method according to claim 1 in which the comparing step includes test samples which lack an associated probability and/or probabilistic weighting and/or in which the comparing step includes another sample results having an associated probability and/or probabilistic weighting and/or in which the comparing step includes another sample results not having an associated probability and/or probabilistic weighting.

12. A method according to claim 1 in which the comparing of the another sample result with the search term considers whether the another sample result equals or is within the search term.

13. A method according to claim 12 in which the another sample result equals or is within the search term by being equal to or within one of more of the parts forming the search term.

14. A method according to claim 5 in which the complying with the requirements of the search term is acknowledged where the another sample result equals or is within the search term and/or not complying with the requirements of the search term is acknowledged where the another sample result does not equals or is not within the search term.

15. A method according to claim 1 wherein a potential match is a match between a test sample result and an another sample result for which the requirements of the search term are complied with and/or a non-match is where another sample result does not comply with respect to anyone of the different identity variable being compared in the search term.

16. A method according to claim 1 in which where one or more potential
matches are identified, the method further includes performing an analysis of a subsequent sample and the method further includes performing one or more of the following steps with respect to the subsequent sample substituted for the test sample or the another sample:
a) the test sample analysis data set is processed by a computer implemented step to give a test sample results data set, the test sample results data set including one or more test sample results, a test sample result including an identity value for one or more of the identity variables considered in the analysis, the possible identity values including a constrained identity value, the constrained identity value being two or more of the possible identity values and less than all of the possible identity values;
b) defining, in a computer implemented step, a search term from one or more of the test sample results, the defining of the search term involving the replacement of one or more of the constrained identity values with an unconstrained identity value, the unconstrained identity value being all the possible identity values;
c) obtaining, in a computer implemented step, the another sample to compare with the test sample, the another sample being in the form of an another sample result data set, the another sample result data set including one or more another sample results, an another sample result including an identity value for one or more of the identity variable considered in an analysis of that another sample;
d) comparing, in a computer implemented step, the another sample result with the search term, the another sample result complying with the requirements of the search term and thereby providing a potential match between the another sample and the test sample or the another sample result not complying with the requirements of the search term and thereby not providing a potential match between the another sample and the test sample;
e) allocating, in a computer implemented step, a probability and/or a probabilistic weighting to one or more of the potential matches;
f) ranking, in a computer implemented step, the one or more potential matches provided with a probability and/or a probabilistic weighting according to the value of the allocated probability and/or probabilistic weighing.

17. A method according to claim 5, in which the test
sample results data set include one or more parts of the test sample analysis data set which are not identity variables, such as non-allelic sources, and/or which are suspected of being caused by sources which are not identity variables, such as non-allelic sources.

18. A method according to claim 5 in which one or more or all of the identity values and/or combinations thereof are provided with a further data element and the further data element indicates one of the following:
an identity value, such as an allele, which is accepted as being an identity value, such as an allele;
an identity value, such as an allele, which is not accepted as an identity value, such as an allele, for instance because it is a not identity variables, such as a non-allelic source;
an identity value, such as an allele, which may or may not be an identity value, such as an allele.

19. A method according to claim 18 in which the further data element is not used in the
search term and/or is not used in the comparing of the test sample and/or is not be used in the determination of potential matches.

20. A method according to claim 18 in which the probability and/or the probabilistic weighting give to a potential match is weighted and the weighting is influenced by the presence of a further data element and/or is set by the presence of a further data element.
